(19) United States
(12) Patent Application Publication
(10) Pub. No.: US 2022/0409263 A1
(43) Pub. Date: Dec. 29, 2022

(54) PLASMA CORING TOOL WITH ENDPOINT DETECTION

(71) Applicant: Medtronic Advanced Energy LLC, Minneapolis, MN (US)

(72) Inventors: Prakash Manley, Arvada, CO (US); Mohammad Miri, Longmont, CO (US); Juan Pablo Angulo, Louisville, CO (US); Xiaoming Cheng, Keller, TX (US); Anjali Dhiman, Commerce City, CO (US); Bradley W. Jacobsen, Erie, CO (US); Wade Schutte, Denver, CO (US); Yeung Chow, Longmont, CO (US)

(21) Appl. No.: 17/849,262

(22) Filed: Jun. 24, 2022

Related U.S. Application Data
(60) Provisional application No. 63/215,215, filed on Jun. 25, 2021.

(51) Int. Cl. A61B 18/14

(52) U.S. Cl. CPC A61B 18/1442; A61B 2018/00083

(57) ABSTRACT

An electrosurgical device including an elongated body extending from a proximal end to a distal end and defining an evacuation lumen. The elongated body including an irrigation channel carried by the elongate body, the irrigation channel configured to deliver a fluid to a target tissue adjacent to the distal end, a coring electrode at the distal end of the elongated body, where the coring electrode defines an opening to the evacuation lumen, and where the coring electrode is configured to operate in a monopolar configuration to deliver radio frequency (RF) plasma energy to adjacent tissue to cut a volume of the target tissue, and a dielectric coating on at least a distal portion of the elongated body, the dielectric coating electrically insulating the elongated body from target tissue and the volume of cut target tissue, where the dielectric coating comprises a ceramic material.

### CROSS-REFERENCE TO RELATED APPLICATIONS

This application claims the benefit of U.S. Application Ser. No. 63/215,215 filed Jun. 25, 2021, entitled “PLASMA CORING TOOL WITH ENDPOINT DETECTION”.

### FIELD

This invention relates generally to surgical methods and apparatuses and particularly to electrosurgical devices.

### BACKGROUND

Electrosurgical devices such as plasma-mediated thermo-electric cutting devices have been developed for use in cutting soft biological tissue in surgical settings. Such devices have found use in various surgical settings and procedures including, but not limited to, spine discectomy and fusion, and other surgical specialties such as general surgery, breast, thoracic, and the like. Typically, such electrosurgical devices are classified as being either monopolar or bipolar electrosurgical devices. A monopolar device generally includes a single electrode carried by the device and configured to communicate with a reference electrode, typically in the form of a return pad, attached to the exterior of a patient. Monopolar electrosurgical devices deliver highly concentrated electrical energy that enhances cutting edges to excise material and then transmits through the tissue of a patient. In contrast, a bipolar electrosurgical device includes a pair of electrodes carried by the device and positioned in close proximity to one another.

### SUMMARY

The techniques of this disclosure generally relate to electrosurgical cutting devices with a monopolar plasma coring tip although a bipolar coring tip is also possible. The disclosed devices may include an irrigation and evacuation system that can be operated in conjunction with the electrosurgical plasma cutting. The irrigation system lowers the temperature of the target tissue and allows overall baseline circuit impedance (sum of saline and target tissue) to stay consistent allowing for more consistent energy delivery to occur during the plasma cutting duration of the procedure as well as provide other benefits as discussed further below. Having a highly insulated electrode with finite exposed cutting edge can permit lower energy application when producing plasma cutting effects. The addition of saline irrigation further helps prevent localized overheating, charring, or damage to the adjacent tissue. This may be particularly useful for certain electrosurgical procedures such as discectomy procedures or those where preservation of delicate tissue and nerves directly adjacent to the cutting site is important for success of the procedure.

In an embodiment, the disclosure describes an electrosurgical device including an elongated body extending from a proximal end to a distal end and defining an evacuation lumen configured to evacuate tissue from the distal end to the proximal end; an irrigation channel carried by the elongate body, the irrigation channel configured to deliver a fluid to a target tissue adjacent to the distal end; a coring electrode at the distal end of the elongated body, where the coring electrode defines an opening to the evacuation lumen, and where the coring electrode is configured to operate in a monopolar configuration to deliver radio frequency (RF) plasma energy to adjacent tissue to cut a volume of the target tissue; and a dielectric coating on at least a distal portion of the elongated body, the dielectric coating electrically insulating the elongated body from target tissue and the volume of cut target tissue, where the dielectric coating comprises a ceramic material.

In another embodiment, the disclosure describes a electrosurgical system including an electrosurgical device including an elongated body extending from a proximal end to a distal end and defining an evacuation lumen configured to evacuate tissue from the distal end to the proximal end; an irrigation channel carried by the elongate body, the irrigation channel configured to deliver a fluid to a target tissue adjacent to the distal end; a coring electrode at the distal end of the elongated body, where the coring electrode defines an opening to the evacuation lumen, and wherein the coring electrode is configured to operate in a monopolar plasma configuration to cut a volume of the target tissue; a dielectric coating on at least a distal portion of the elongated body, the dielectric coating electrically insulating the elongated body from target tissue and the volume of cut target tissue, where the dielectric coating includes a ceramic material; a return electrode; and a power supply coupled to the electrosurgical device and reference electrode, where the power supply is configured to deliver radio frequency (RF) plasma energy in of at least about 100V to the coring electrode to cut a volume of the target tissue.

In another embodiment, the disclosure describes a method of producing a coring electrode for an electrosurgical device including providing an elongate body that includes a metal substrate having a beveled distal end, where the elongated body has an inner surface and an outer surface, the inner surface defining an evaluation lumen that extends from the distal end to a proximal end of the elongated body; and coating a distal portion of the elongated body to apply with a ceramic material to form a dielectric coating on the inner and the outer surfaces of the elongated body, where the metal substrate at the beveled distal end is sufficiently exposed to define a coring electrode configured to deliver radio frequency (RF) plasma energy in the range of about 200 kHz to about 3.3 MHz to adjacent tissue in a wet field monopolar configuration.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The disclosure can be more completely understood in consideration of the following detailed description of various embodiments of the disclosure, in connection with the accompanying drawings, in which:

FIG. 1 is a schematic view of an example electrosurgical device that includes a coring electrode tip.

FIGS. 2A and 2B are schematic cross-sectional views of a distal portion of the elongated body illustrating cutting features of the coring electrode of the electrosurgical device of FIG. 1.

FIG. 3 is schematic view of another distal portion of an elongated body that includes a coring electrode and a depth gauge that may be used with the electrosurgical device of FIG. 1.

FIGS. 4A and 4B are schematic views of another electrosurgical device that includes a coring electrode and an articulating tip.

FIG. 5 is a flow diagram of an example method of producing the coring electrode of FIG. 1.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

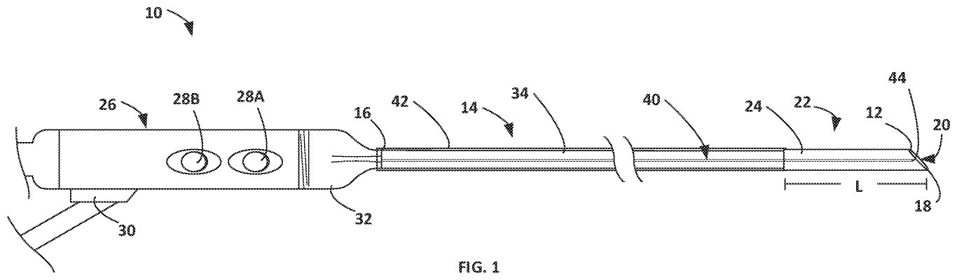

FIG. 1 is a schematic view of an example electrosurgical device 10 as described herein that includes a coring electrode 12 tip that may be used with electrosurgical procedures including, but not limited to, performing a discectomy or other procedures where heat generation or preservation of delicate adjacent tissue are important. A discectomy procedure involves the surgical removal of an intervertebral disc and fusion of adjacent vertebra. Intervertebral discs are flexible pads of fibro cartilaginous tissue tightly fixed between the vertebrae of the spine. The discs comprise a flat, circular capsule roughly 1 to 2 inches in diameter and about 0.25 to 0.5 inches thick and made of a tough, fibrous outer membrane called the annulus fibrosus, adjacent an elastic core called the nucleus pulposus. Under stress, it is possible for the annulus fibrosus to fail or the nucleus pulposus to swell and herniate, pushing through a weak spot in the annulus fibrosus membrane of the disc and into the spinal canal. Consequently, all or part of the annulus fibrosus and/or nucleus pulposus material may protrude through the weak spot, causing pressure against adjacent nerves which results in pain and immobility.

Where a damaged intervertebral disc must be removed from the patient as part of a discectomy and a subsequent fusion of vertebral bodies of the superior and inferior vertebrae, the surgeon may first retract soft tissue from the point of entry to the vertebrae to be fused. Around and attached to the vertebrae are, among other things, various muscles which act on the vertebrae to affect movement of the upper body. Once the retraction is complete, and the disc is exposed, the disc may be removed. The vertebrae may then be aligned to straighten the spinal column, and stabilized relative to one another by rods or other supports which are attached to the vertebrae by numerous fastening techniques. The surgeon may then place implants and bone grafts across the exposed surfaces of adjoining vertebrae and restore the location of the soft tissue to cover the bone graphs and vertebrae. The grafts regenerate, grow into bone and fuse the vertebrae together, with the implant and rod functioning as a temporary splint which stabilizes the spinal column while the bone fuses together over a period of months.

During the discectomy and fusion, the disclosed devices may be particularly useful to separate and remove the intervertebral disc without damage to the adjacent tissue and nerve root. The disclosed devices may be configured to operate in conjunction with an irrigation system to generate low impedance environment that may help reduce the heat generation during the plasma cutting to cut and separate the target tissue without generating excessive heat, charring, or otherwise negatively impacting the adjacent tissue. In some examples, the devices may also be used to shrink and seal blood vessels of the vertebral venous or arterial systems against blood loss before or after the vessels are cut, rupture or are otherwise severed.

Electrosurgical device 10 includes an elongated body 14 extending from a proximal end 16 to a distal end 18. Elongated body 14 may define an inner lumen configured to evacuate and remove excised tissue from distal end 18 toward proximal end 16 (evacuation lumen 20). Distal end 18 defines coring electrode 12 which is configured to cut adjacent tissue using plasma energy. In some examples, to form coring electrode 12, the distal portion 22 of elongated body 14 may include a dielectric coating 24 configured to electrically insulate distal portion 22 (e.g., apart from coring electrode 12) of elongated body 14 from the adjacent patient tissue, the excised material being evacuate through evacuation lumen 20, or both. For example, elongated body 14 may include an electrically conductive substrate 34 (e.g., stainless steel) such that the entire elongated body 14 acts as the electrical conductor for coring electrode 12. Dielectric coating 24 may include a ceramic material applied to the inner and outer sidewalls of elongated body 14 with coring electrode 12 being defined by the exposure of underlying electrically conductive substrate 34 at distal end 18. In such examples, dielectric coating 24 electrically insulates distal portion 22 of substrate 34 from adjacent patient tissue that might otherwise contact the surface of the inner or outer sidewalls to conduct current and dissipate energy into tissue, results in undesired tissue effect and low cutting performance on target tissue.

Electrosurgical device 10 may also include a handle assembly 26, which in turn is configured to couple to an electrosurgical power supply (not shown) that delivers the electric energy to coring electrode 12. The electrosurgical power supply may be configured to generate and provide radiofrequency (RF) monopolar energy with a power curve having its power peak at low impedance range, designed for cutting under saline. Handle assembly 26 may also include one or more switches or buttons 28A and 28B for activating coring electrode 12 to deliver or adjust the desired electrosurgical energy to the adjacent tissue, to control irrigation, or to initiate suction for excavation of material through evacuation lumen 20. Additionally, or alternatively, handle assembly 26 can include a stand or mount for stabilizing device 10 during an electrosurgical procedure. Handle assembly 26 may also include other switches or buttons for actuating other features of device 10, additional connectors for coupling device 10 to other components (e.g., coupler 30 for connecting to negative pressure pump and reservoir for excavation of material) during the procedure, and the like.

In some examples, elongated body 14 may be detachably coupled to handle assembly 26. For example, proximal end 16 of elongate body 14 may be attached to a connector 32 (e.g., screw, snap, or friction fit connector) that can be easily attached and detached from handle assembly 26 to allow for easy cleaning of elongated body 14 or interchange with other similarly configured elongated bodies having different geometries or configurations (e.g., larger diameter coring electrode, alternative bevel angles, different coring electrode shapes, or the like). Connector 32 may be sufficiently sized for a clinician to grasp and detach elongated body 14 while in an operating room. In such examples, connector 32 should be configured such that attachment to handle assembly 26 provides proper coupling between coring electrode 12 and the power source, connection to the irrigation and suction assemblies, coupling of any articulation mechanism (if present), sensor elements, and the like.

Coring electrode 12 is carried by distal portion 18 of elongated body 14 and configured to deliver electric energy (e.g., RF plasma, including a pulsed electron avalanche plasma, or ablation energy) to adjacent patient tissue (e.g., soft tissue or disc material) to cut a volume of the tissue as said volume is conveyed into evacuation lumen 20. In some examples, the opening that provides entry to evacuation lumen 20 may be defined at least in part by the geometry of coring electrode 12. As electric energy is delivered to the adjacent soft tissue, coring electrode 12 cuts the adjacent tissue to create a volume of tissue (e.g., excised tissue) that enters through the opening defined by coring electrode 12 and is conveyed into evacuation lumen 20.

Coring electrode 12 may be composed of any suitable conductive material including, but not limited to, stainless steel, titanium, platinum, iridium, niobium or alloys thereof. As described above, distal portion 22 of elongated body 14 may be coated with dielectric coating 24 such that distal end 18 of elongated body 14 is exposed to the adjacent tissue or includes a non-substantial amount of dielectric coating 24 such that the exposed portion define coring electrode 12.

Dielectric coating 24 may include a ceramic material that produces an electrical barrier between underlying conductive substrate 34 of elongated body 14 and adjacent tissue. Dielectric coating 24 may be applied to both the inner and outer surfaces of elongated body 14 followed by, if needed, sintering of the ceramic material to produce a non-porous dialectic coating. The inclusion of a ceramic (e.g., glass) dielectric coating ensures that the coating is capable of withstanding the high temperatures that can be produced by coring electrode 12 (e.g., possible temperatures in excess of 1000° C.) without melting, delaminating, or otherwise physically or electrically degrading during operation.

Any suitable ceramic material may be used to produce dielectric coating 24 provided the material can produce a film coating that has a dielectric strength of at least about 1000 V, e.g., at least 3000 V. Dielectric coating 30 may have a thickness of about 2-4 mils (e.g., about 0.05 mm to about 0.1 mm). In preferred examples, the ceramic material should be selected to have a comparable coefficient of thermal expansion (CTE) with substrate 34 of elongated body 14 (e.g., stainless steel). For example, because the operation of coring electrode 12 can produce a large temperature gradient on the order of 1000° C. between distal end 18 and other portions of elongated body 14 along with the rapid cooling effects generated by irrigation system 40, large discrepancies between the CTE of substrate 34 and dielectric coating 24 can generate mechanical stress at the interface between substrate 34 and dielectric coating 24 which in turn can cause the coating to fail due to spallation, cracking, and the like. By selecting a dielectric coating 24 that possesses a CTE similar to that of substrate 34 (e.g., CTE values within ±10% of one another) ensures that mechanical stress along the interface between dielectric coating 24 and substrate 34 is sufficiently low to avoid one or more of the above-described complications.

Suitable ceramic materials that may be used to produce dielectric coating 24 may include, but are not limited to, alumina, zirconia, alkaline borosilicate glass, alkaline earth borosilicate glass, silicate glass-ceramics and the like. In some examples where substrate 34 is stainless steel (CTE of approximately 11), dielectric coating 24 may include alkali and alkaline earth borosilicate glasses. Additionally, or alternatively, the ceramic material selected for dielectric coating 24 should be medically safe or an inert material.

Dielectric coating 24 may be applied over the entire length of elongated body 14 or only a set length (L) of distal portion 22. While in general, it may be preferable to electrically insulate the entire exterior and interior surface of substrate 34, the extreme temperature fluctuations during operation may be localized to portions adjacent to distal end 18. Thus, dielectric coating 24 comprising the ceramic material may extend over only length (L) of distal portion 22 along the inner and outer surfaces of substrate 34. The remaining exterior surface of substrate 34 may be covered with a second dielectric material 42, such as a shrink-wrap polymeric material that is relatively inexpensive in terms of material and manufacturing costs while providing the desired dielectric characteristics. The second dielectric material 42 may have a relatively low melting or failure temperature that would otherwise make the material unsuitable if used within distal portion 22 near coring electrode 12. However, due to the separation distance (L) between the second dielectric material 42 and coring electrode, the localized temperature of substrate 34 may remain relatively low and within the operational parameters of second dielectric material 42. Additionally, or alternatively, second dielectric material 42 may be used to secure other components to substrate 34 such as one or more irrigation conduits 40, electrical conductors, actuating levers, and the like.

Other components of device 10 can be fabricated from biologically acceptable materials suitable for medical applications, including but not limited to, electrically conductive metals, synthetic polymers, ceramics, and combinations thereof. Some such materials may include metals such as stainless steel alloys and titanium, thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), semi-rigid and rigid materials, elastomers, rubbers, thermoplastic elastomers, thermoset elastomers, elastomeric composites, rigid polymers including polyphenylene, polyamide, polyimide, polyetherimide, polyethylene, and combinations thereof.

Coring electrode 12 may be configured as a monopolar electrode configured to provide low energy RF plasma. For example, coring electrode 12 may communicate with a reference electrode (not shown) such as a back plate, dispersive pad, or topical pad connected to electrosurgical power supply to provide a monopolar arrangement. As used herein, the term “reference electrode” is used to signify an electrode configured to communicate with coring electrode 12 in a monopolar arrangement and is itself not carried by elongated body 14. Coring electrode 12 may be calibrated to deliver relatively low-level electric energy in the form radio frequency or pulsed radio frequency energy delivery to produce plasma used to cut through adjacent soft tissue.

In a monopolar electrosurgical configuration, the active electrode, such as coring electrode 12, is positioned at the target surgical site. The reference electrode may be placed somewhere on the patient's body. Electrical current passes through the patient as it completes the electrical circuit from the active electrode to the reference electrode. The reference electrode has a much larger conductive surface area compared to the active electrode to help safely dissipate the electrical energy and prevent localized heating. In contrast, the active electrode has a much smaller surface area allowing for plasma to be produced at the treatment site to produce the desired cutting effect. The electric current may be concentrated in the area of contact of the active electrode offering versatility and function with a variety of electrosurgical waveforms to produce different tissue effects.

A conventional monopolar electrosurgical configuration involves dry field plasma cutting that typically involves relatively high energy levels (e.g., an amplitude of at least about 100V) to generate plasma at the contact point of the electrode. The relatively high energy levels can cause charring or increased localized heating that may detrimentally impact sensitive adjacent tissue. Additionally, such charring of the adjacent tissue can significantly increase the impedance level at the point of contact thereby diminishing the effectiveness of the plasma generation.

The low impedance plasma generation of the present electrosurgical device 10 may be obtained by providing continuous irrigation at the site of contact between coring electrode 12 and adjacent patient tissue. The continuous irrigation lowers the impedance level observed between coring electrode 12 and the return electrode. The continuous irrigation can help cool the target tissue as well as the surface temperature of coring electrode 12 and other parts of device 10. The irrigation also provides the ability to have a consistent impedance environment for the application of energy. The cooling effect can reduce the localized heating of target tissue during the electrosurgical procedure as well as significantly reduce the charring affects to the adjacent tissue thereby helping to preserve delicate adjacent tissue.

In some examples the plasma energy may be produced by, low voltage, current, power and/or low duty cycle waveforms. Low power waveforms may refer to low voltage, continuous waveforms. Low duty-cycle may refer to the proportion of time that the energy is actually being applied and may include cycles of less than 10% which may be, for instance, 1% or less, or 0.1% or less. A pulsed low duty-cycle signal may include a plurality of pulse bursts that are separated by more than one millisecond (e.g., has a frequency of less than 1 kHz) where each burst is shorter than one millisecond which may assist in minimizing tissue charring or burning. In some examples, the overall circuit impedance resulting from the introduction of irrigation may be on the order of about 150 to about 600 Ω. The low-level impedance may in turn allow for coring electrode 12 to be calibrated to a low-electrical signals suitable to create a plasma in the range of about 200 kHz to about 3.3 MHz applied in continuous, burst, or pulsed waveforms having an amplitude of at least about 100V. Each burst typically has a duration in the range of 10 microseconds to 1 millisecond with each burst having a duty duration of about 0.1 to 10 microseconds. The pulses may be bi-phasic square waves that alternate positive and negative amplitudes. The interval between pulses should be shorter than a lifetime of the plasma vapor cavity in order to maintain the cavity and the plasma regime during each pulse burst. The time between the pulse bursts should be sufficient so that the duty-cycle remains relatively low thereby helping to minimize undesirable heating effects. Alternatively, the pulses may be a continuous sine wave of the frequency described above.

Coring electrode 12 may define a cutting edge of device 10. In some examples, the cutting edge may be mechanically sharp and produced by incorporating a beveled edged having a tip angle (α) as measured orthogonal to a central axis of elongated body 14. Accordingly, a blunt tip would represent beveled angle (α) of 0°. In some examples, the beveled angle (α) may be about 10° to about 45°, or more preferably about 15° to about 25°. Additionally or alternatively, the distal end may also include a compound beveled edge such that in addition to the beveled angle (α), the cutting edge include an additional bevel from the outside of elongated body 14 toward the center. Including the mechanically sharp tip at distal end 18 may also help ensure proper exposure of the surface of substrate 34 during the application of dielectric coating 24 to ensure proper definition and operation of coring electrode 12. For example, the dielectric coating 24 may be formed by sintering of ceramic particles. After sintering, the cutting edge is sufficiently exposed to provide definition of coring electrode 12 while creating a sufficient dielectric coating 24 on the adjacent portions of substrate 34.

Electrosurgical device 10 is also configured to provide both evacuation and irrigation at the treatment site. For example, elongated body 14 may include one or more irrigation channels 40 configured to convey a fluid (e.g., saline) from handle assembly 26 to one or more exit orifices 44 positioned in close proximity to distal end 18 (e.g., within about 2 mm to 5 mm of the cutting tip) such that the fluid is delivered to the treatment site during the electrosurgical procedure in a manner to allow the site to be cooled before the fluid is evacuated from the site. To help provide fluid connection, handle assembly 26 may be coupled to a fluid delivery system and connector 32 may be configured to provide a fluid connection between handle assembly 10 and the fluid delivery system and irrigation channels 40. In some examples, the preferred flow rate for delivery of a fluid to the target delivery site may be on the order of about 10 ml/min to about 55 ml/min. Additionally, the irrigation may be actuated by the clinician using one of switches 28A or 28B or may be tied with the activation of the RF energy signal.

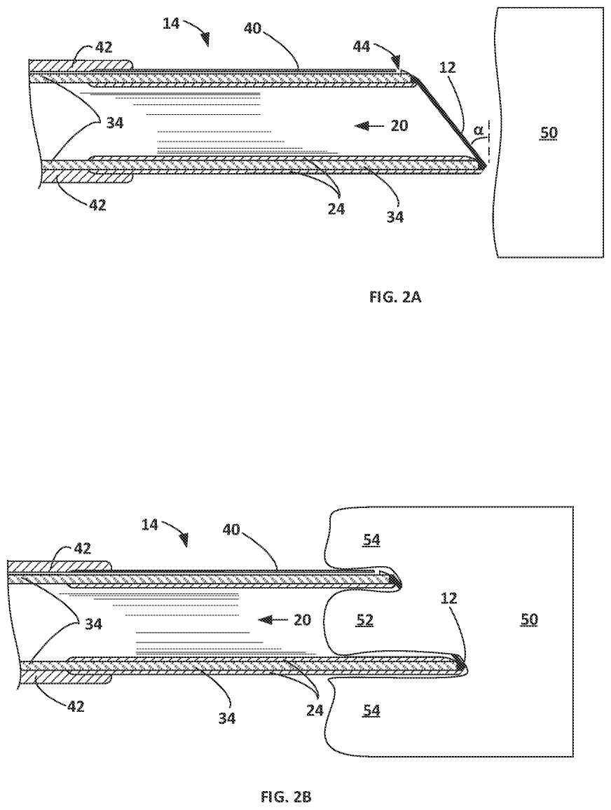

FIGS. 2A and 2B are schematic cross-sectional views of distal portion 22 illustrating cutting features of coring electrode 12 during operation. The configuration of coring electrode 12 defines an opening that provides entry into evacuation lumen 20. As electric energy is delivered to the adjacent soft tissue 50, coring electrode 12 cuts the adjacent tissue 50 to create a volume of tissue (e.g., excised tissue 52) that enters through the opening defined by coring electrode 12 and is conveyed into evacuation lumen 20 using a negative pressure system. Irrigation channels 40 and exit orifices 44 provide fluid saturation to the target treatment site to ensure cooling and relatively constant impedance with soft tissue 50 while coring electrode 20 forms excised tissue 52. Excised tissue 52 may be separated from tissue 50 by various means including briefly pausing forward motion and allowing the energy to cut across the inner radius of electrode 12 or slightly tilting or transversely moving electrode 12 to cut the base of excised tissue 52. The adjacent tissue 50 is preserved and passes along the exterior of elongated body 14 with minimal heat generation, charring, or damage.

To help facilitate conveyance of excised tissue 52 along evacuation lumen 20, handle assembly 26 is coupled to a negative pressure source to provide suction and collection of excised material 52. The continuous irrigation of the treatment site and removal of such fluid via evacuation lumen 20 may help lubricate and encourage the passage of excised material 52 into evacuation lumen 20. Additionally, to help facilitate removal of excised tissue 52, the surface of evacuation lumen 20 may be coated or treated to prevent tissue adherence. Such coatings may include lubricious silicone materials or repellant omniphobic materials. Such treatments may include hydrophobic surface patternings.

The inner and outer diameter of elongated body 14, and more specifically distal end 18, may be any suitable size and may be tailored for a specified procedure. For example, the outer diameter may be on the order of about 3 to about 7 mm. Having the outer diameter of distal end 18 be less than about 4 mm may be particularly suited for discectomy procedures to provide sufficient access during posterior lumbar interbody fusion (PLIF) or transforaminal lumbar interbody fusion (TLIF) surgery. In some examples, the inner diameter elongated body 14 (e.g., diameter of evacuation lumen 20) may be about 2 mm to about 6 mm to ensure proper excavation of excised material 52 without producing an obstruction.

The cross-section of elongated body 14 may take on any suitable shape and size as desired for particular applications. For example, elongated body 14 may possess a tubular body having a circular, semi-circular, oval, curvilinear, rectangular, trapezoidal, triangular, or some other multi-faceted cross-sectional shape. In some examples, it may be useful have a combination of curved and straight sides to provide the clinician with multiple edging options for excising tissue. Further the intersections between adjacent sides may themselves be curvilinear, abrupt resulting in distinct edge transitions, or a combination of curvilinear and abrupt transitions. Additionally, or alternatively, the distal end of elongated body may include a curette shape (e.g., shovel or spoon shaped) to assist with certain procedures. The selection of cross-sectional shape of elongated body 14, and by association coring electrode 12, may help simultaneously improve tissue removal and tissue preservation in treatment areas having particular size or shape constraints or delicate adjacent tissue that can be easily damaged.

In some examples, coring electrode 12 or alternatively another external electrode carried by elongated body 14 (not shown) may also be configured for use in a sensing capacity to measure or interrogate one or more properties of adjacent tissue 50. Such sensing may include measuring the impedance of the adjacent tissue 50 to determine if there is a significant change to the type of tissue (e.g., contact with bone or other tissue density than desired), the properties of the tissue (e.g., charring of the tissue), or complications with the system (e.g., a slow or loss of irrigation). The sensing capacity can enhance the safety capacity of device 10 by providing more accurate feedback of the adjacent tissue during use.

In some examples, electrosurgical device 10 may include or be configured to receive a camera to view and monitor progress of excised tissue 52 from the target treatment site. For example, handle assembly 26 and evacuation lumen 20 may be configured to decouple from the negative pressure source and receive a borescope that is traversed through evacuation lumen 20 toward distal end 18 to inspect the target treatment site. After inspection, the borescope can be removed and the negative pressure source reattached to continue the electrosurgical procedure.

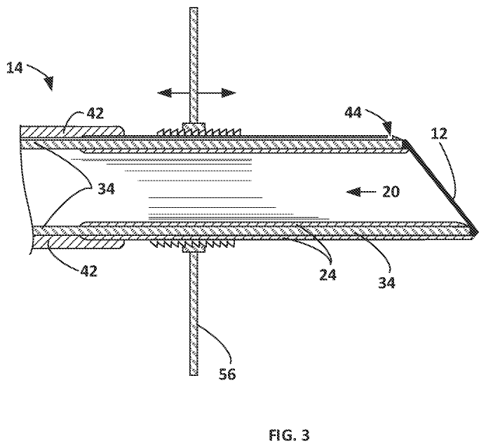

Electrosurgical device 10 may also include an adjustable depth gauge configured to either indicate or prevent further axial movement when distal end 18 has entered into soft tissue 50 to a set depth. For example, FIG. 3 is a schematic cross-sectional view of example elongated body 14A that includes an adjustable depth gauge 56. Depth gauge 56 may be attached to the exterior surface of elongated body 14A (e.g., exterior relative to dielectric coating 24 such that depth gauge 56 does not interact or impede the delivery of plasma energy to tissue 50 by coring electrode 12). Depth gauge 56 may include a disc shaped body orthogonal to the central axis of elongate body 14A. The disc-shaped body can act as a physical stop for elongated body 14A preventing further distal movement into target tissue 50 during an electrosurgical procedure after reaching the preset depth. The depth gauge may be made of a transparent material so as to not visually obstruct the treatment site.

In some examples, depth gauge 56 may be adjustable by a turn screw assembly allowing for precise depth adjustment relative to the number of rotations about elongated body 14. Alternatively, depth gauge 56 may be friction fitted (e.g., silicone sheath) over elongated body 14 allowing for sufficient resistance once the preset depth of cutting electrode 12 has been reached while still permitting the clinician to slide depth gauge 56 to a desired depth.

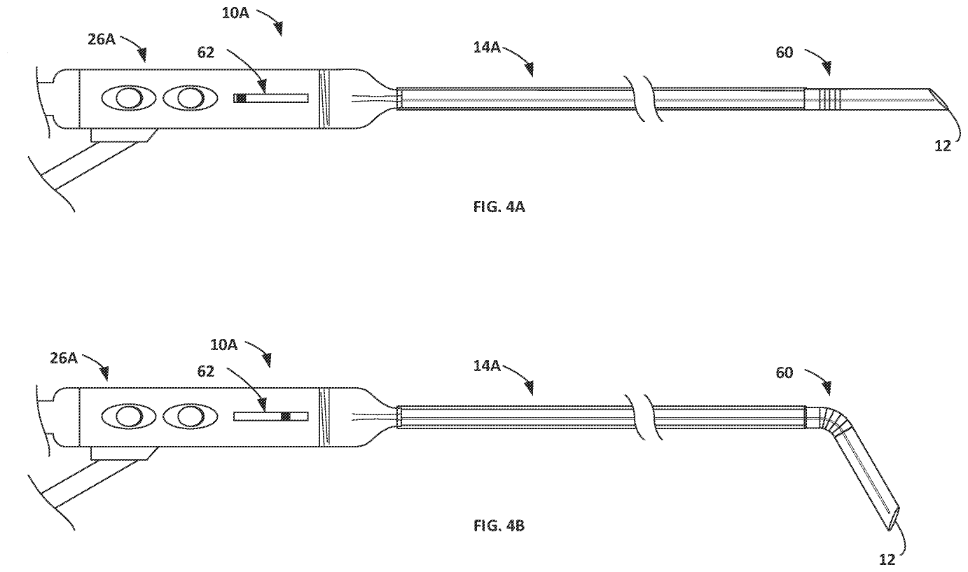

FIGS. 4A and 4B are schematic views of another electrosurgical device 10A having coring electrode 12 as described above with an articulating elongated body 14A. The articulation point 60 may be positioned proximal of the distal end and coring electrode 12 by a set distance tailored to a desired procedure (e.g., about 12 mm to about 65 mm from the distal end for discectomy procedures). Articulation point 60 may be actuated through handle assembly 26A through slider mechanism 62. The articulating tip may allow the clinician to steer the distal end of elongated body 14A to allow coring electrode 12 to reach restrictive areas at the target treatment site without needing a direct line of sight access to the treatment location. Such articulation may be particularly useful for certain types of clinical procedures such as a discectomy. For example, in discectomy procedure, the point of entry to the fibro cartilaginous tissue may be obtained through minimally invasive TLIF or PLIF incision access. With a straight electrosurgical device, full removal of the fibro cartilaginous tissue may be impeded unless access is provided through multiple points of entry. Even then, full removal of fibro cartilaginous tissue against the proximal wall of the spine may be limited. Having an articulating tip may substantially improve removal of fibro cartilaginous tissue and possibly reduce the number of access points needed to complete a particular procedure.

Articulation point 60 may be designed and implemented in a substantially similar manner to the articulation systems used with catheter assemblies, optical probes, and the like. Examples of articulation mechanisms for elongated bodies that may be incorporated into electrosurgical device 10 are described in U.S. Pat. No. 10,039,532 B2 entitled “Surgical Instrument with Articulation Assembly” by Srinivas et al.; U.S. Pat. No. 10,660,623 B2 entitled “Centering Mechanism for Articulation Joint” by Nicholas; U.S. Pat. No. 10,561,419 B2 entitled “Powered End Effector Assembly with Pivotable Channel” by Beardsley; and U.S. Pat. No. 8,403,946 B2 entitled “Articulating Clip Applier Cartridge” by Whitfield et al. all of which are incorporated by reference in their entirety. In other embodiments, electrosurgical device 10 may include a straight, bent, curvilinear, or other shaped elongate body 14 that is permanently shaped in such manner.

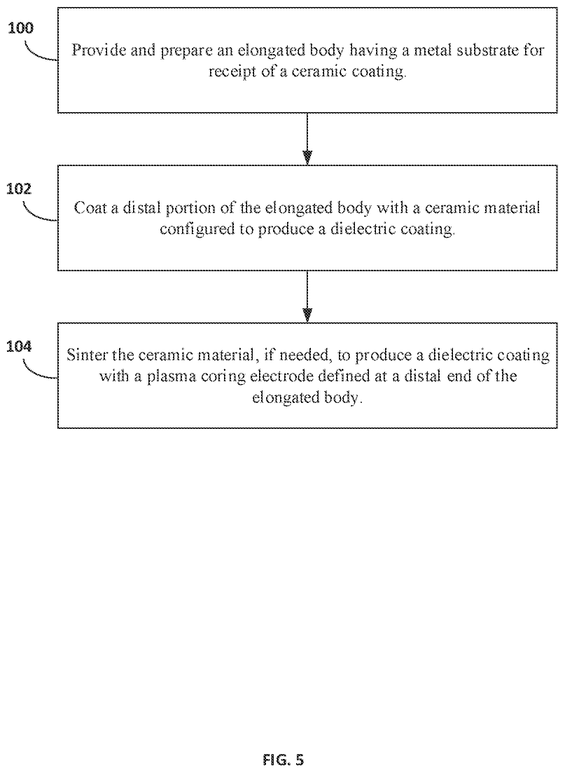

FIG. 5 is a block diagram of an example technique of producing coring electrode 12 on an electrosurgical device 10. The below description is described with respect to electrosurgical device 10 of FIG. 1. However, the disclosed technique may be used to produce other electrosurgical devices or other techniques may be used to produce electrosurgical device 10.

The technique of FIG. 5 includes providing and preparing an elongated body 14 having a metal substrate for receipt of ceramic coating (100), coating a distal portion 22 of elongated body 14 with a ceramic material configured to produce a dielectric coating (102), and, if needed, sintering the ceramic coating to produce a dielectric coating 24 with a coring electrode 12 defined at the distal end 18 of elongated body 14 (104).

As described above, elongated body 14 may be formed from an electrically conductive metal substrate 34 such as a metal tube. The substrate should consist of a metal material capable of conducting the electrical signals necessary for producing the described RF plasma energy without excessive resistance or heat generation within the substrate 34 itself. Suitable materials may include, but are not limited to, various grades and hardness of stainless steel.

Substrate 34 may be prepared by providing an initial bevel cut at distal end 18. The bevel cut may provide a sharp leading edge that defines the surface area and location for coring electrode 12. The leading edge may be a chiseled edge based on the bevel angle or may be further sharpened as desired by to incorporating a compound bevel, convex or hollow edge, v-edge, or the like. The relative sharpness of the cutting edge may also help mechanically excise target tissue in addition to the electrosurgical cutting effects produced by coring electrode 12.

To help assist with the application of dielectric coating 24, distal portion 22 of substrate 14 optionally may be initially cleaned, chemically etched or treated, or the like. Such treatment may help ensure proper adherence of the resultant dielectric coating 24 thereby reducing the likelihood of delamination, cracking, spallation, or other defects between substrate 34 and dielectric coating 24.

Once prepped, distal portion 22 may be coated a ceramic material configured to produce dielectric coating 24 (102). Any suitable technique may be used to apply the ceramic material that produces dielectric coating 24. Suitable ceramic materials that may include non-toxic materials such as borosilicate glass having a relatively high or upon sintering provide a coating with a relatively high dielectric constant.

While the portion of substrate 34 receiving the coating application and thereby dielectric coating 24 may be any suitable length including the entire length of substrate 34, the coating should be applied to substrate for a length of about 35 mm to about 50 mm of the body measured from the distal end 18. Having the coating applied over at least such a length can help ensure proper electrical insulation between coring electrode 12 and adjacent tissue including excised tissue 52. While the outer surface of elongate body 14 may also receive a second dielectric coating 42, such a coating may not be configured to withstand the localized high temperatures near coring electrode 12, hence the minimal length of dielectric coating 24 can ensure the presence of a dielectric coating 24 near distal end 18 that can withstand the large temperature fluctuations.

The method of FIG. 5 includes, if needed, sintering the coating to produce a dielectric coating 24 with a coring electrode 12 defined at the distal end 18 of elongated body 14 (104). The sintering process may include heating substrate 34 to coalesce ceramic coating to form a non-porous coating. The resulting dielectric coating 24 should have a dielectric strength of 1000V/mil minimum.

Various embodiments of systems, devices, and methods have been described herein. These embodiments are given only by way of example and are not intended to limit the scope of the claimed inventions. It should be appreciated, moreover, that the various features of the embodiments that have been described with respect to the different figures may be combined in various ways to produce numerous additional embodiments. For example, variations of the different electrodes may be combined with other internal electrodes, coring electrodes, external electrodes, and combinations thereof to produce an electrosurgical device tailored for a particular application or procedure. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. An electrosurgical device comprising:
an elongated body extending from a proximal end to a distal end and defining an evacuation lumen configured to evacuate tissue from the distal end to the proximal end;
an irrigation channel carried by the elongate body, the irrigation channel configured to deliver a fluid to a target tissue adjacent to the distal end;
a coring electrode at the distal end of the elongated body, wherein the coring electrode defines an opening to the evacuation lumen, and wherein the coring electrode is configured to operate in a monopolar configuration to deliver radio frequency (RF) plasma energy to adjacent tissue to cut a volume of the target tissue; and
a dielectric coating on at least a distal portion of the elongated body, the dielectric coating electrically insulating the elongated body from target tissue and the volume of cut target tissue, wherein the dielectric coating comprises a ceramic material.

2. The electrosurgical device of claim 1, and wherein the coring electrode is configured to provide (RF) plasma energy in the range of about 200 KHz to about 3.3 MHz.

3. The electrosurgical device of claim 1, wherein elongated body comprises a tubular body having an external surface and an interior surface defining the evacuation lumen, wherein the dielectric coating is applied to both the external surface and the interior surface over a length of the elongated body.

4. The electrosurgical device of claim 3, wherein the length over which the dielectric coating is applied is between about 35 mm to about 50 mm from the distal end of the elongated body.

5. The electrosurgical device of claim 1, wherein the tubular body comprises an electrically conductive metal, and wherein exposure of the conductive metal at the distal end defines the coring electrode.

6. The electrosurgical device of claim 1, wherein the dielectric coating has a coefficient of thermal expansion of about 8 ppm to about 15 ppm and a dielectric strength of at least about 1000V.

7. The electrosurgical device of claim 1, wherein the dielectric coating has a coefficient of thermal expansion that is within ±10% of a coefficient of thermal expansion of a metal substrate forming the elongated body.

8. The electrosurgical device of claim 1, wherein the dielectric coating comprises alkaline earth borosilicate glass.

9. The electrosurgical device of claim 1, wherein the dielectric coating comprises a non-porous film configured to withstand temperatures of at least about 800° C. without melting.

10. The electrosurgical device of claim 1, further comprising a second dielectric coating applied over an exterior surface of the elongated body, the second dialectic coating at least partially overlapping the dielectric coating on the portion of the elongated body.

11. The electrosurgical device of claim 10, wherein the second dielectric coating affixes the one or more irrigation channels to the elongated body.

12. The electrosurgical device of claim 1, wherein the coring electrode is set at a bevel angle of about 10° to about 45° as measured from a longitudinal access of the elongated body.

13. The electrosurgical device of claim 1, wherein the elongated body comprises a cutting edge that defines the coring electrode, wherein the cutting edge comprises at least one of a compound bevel, convex bevel, hollow bevel, or v-edge bevel.

14. The electrosurgical device of claim 1, wherein the elongated body is configured to receive a borescope through the evacuation lumen for visualization of the target treatment site.

15. An electrosurgical system comprising:
an electrosurgical device comprising:
an elongated body extending from a proximal end to a distal end and defining an evacuation lumen configured to evacuate tissue from the distal end to the proximal end;
an irrigation channel carried by the elongate body, the irrigation channel configured to deliver a fluid to a target tissue adjacent to the distal end;
a coring electrode at the distal end of the elongated body, wherein the coring electrode defines an opening to the evacuation lumen, and wherein the coring electrode is configured to operate in a monopolar plasma configuration to cut a volume of the target tissue;
a dielectric coating on at least a distal portion of the elongated body, the dielectric coating electrically insulating the elongated body from target tissue and the volume of cut target tissue, wherein the dielectric coating comprises a ceramic material;
a return electrode; and
a power supply coupled to the electrosurgical device and reference electrode, wherein the power supply is configured to deliver radio frequency (RF) plasma energy in of at least about 100V to the coring electrode to cut a volume of the target tissue.

16. The electrosurgical system of claim 15, further comprising a negative pressure source coupled to the electrosurgical device, the negative pressure source configured to draw and collect tissue from the proximal end to the distal end of the elongated body.

17. The electrosurgical system of claim 15, further comprising an irrigation system coupled to the electrosurgical device, the irrigation system configured to deliver a conductive fluid through the irrigation channels to saturate the target treatment site.

18. A method of producing a coring electrode for an electrosurgical device, the method comprising:
providing an elongate body comprising a metal substrate having a beveled distal end, wherein the elongated body comprises an inner surface and an outer surface, the inner surface defining an evaluation lumen that extends from the distal end to a proximal end of the elongated body; and
coating a distal portion of the elongated body to apply with a ceramic material to form a dielectric coating on the inner and the outer surfaces of the elongated body, wherein the metal substrate at the beveled distal end is sufficiently exposed to define a coring electrode configured to deliver radio frequency (RF) plasma energy in the range of about 200 kHz to about 3.3 MHz to adjacent tissue in a wet field monopolar configuration.

19. The method of claim 18, wherein the dielectric coating has a coefficient of thermal expansion of about 8 ppm to about 15 ppm and a dielectric strength of at least 1000V.

20. The method of claim 18, wherein the dielectric coating has a coefficient of thermal expansion that is within ±10% of a coefficient of thermal expansion of a metal substrate forming the elongated body.

21. The method of claim 18, further comprising forming a beveled edge on the distal end of the elongated body, wherein the beveled edge defines a bevel angle of about 10° to about 45°.

22. The method of claim 18, further comprising applying a second dielectric coating over the external surface of the elongated body, wherein the second dielectric coating overlaps with the dielectric coating and a distal end of the second dielectric coating is at least about 10 mm away from the coring electrode.
